(19) United States
(12) Patent Application Publication
(10) Pub. No.: US 2026/0335664 A1
(43) Pub. Date: Oct. 29, 2026

(54) IMPLANTABLE MEDICAL ELECTRICAL LEAD FOR EXTRAVASCULAR ELECTRICAL STIMULATION

(71) Applicant: Medtronic, Inc., Minneapolis, MN (US)

(72) Inventors: Vladimir P. Nikolski, San Diego, CA (US); Mark T. Marshall, Cape Coral, FL (US); William J. Clemens, Fridley, MN (US); Dina L. Williams, Andover, MN (US); Matthew J. Hoffman, St. Paul, MN (US)

(21) Appl. No.: 18/997,328

(22) PCT Filed: Jul. 17, 2023
(86) PCT No.: PCT/US2023/027899

§ 371 Date: Jan. 21, 2025

Related U.S. Application Data
(60) Provisional application No. 63/369,779, filed on Jul. 29, 2022.

(51) Int. Cl. A61N 1/39; A61N 1/05

(52) U.S. Cl. CPC A61N 1/39622; A61N 1/05

(57) ABSTRACT

An example implantable medical electrical lead includes a lead body defining a proximal portion and a distal portion, wherein at least a part of the distal portion of the lead body defines a three-dimensional undulating configuration; a. proximal end of the distal portion disposed adjacent to the three-dimensional undulating configuration; a first defibrillation electrode and a second defibrillation electrode disposed along the three-dimensional undulating configuration spaced apart from one another, and a pacing electrode disposed between the first defibrillation electrode and the second defibrillation electrode. The first defibrillation electrode is disposed between the proximal end and the pacing electrode. A first portion of tire first defibrillation electrode, a first portion of the second defibrillation electrode, and the pacing electrode are disposed along a plane. A second portion of the first defibrillation electrode adjacent to the pacing electrode arcs orthogonally to the plane.

This application is an international application with provisional priority of U.S. Provisional Patent Application No. 63/369,779, filed 29 Jul. 2022, the entire contents of which are incorporated herein by reference.

### TECHNICAL FIELD

The present application relates to implantable medical leads and, more particularly, implantable medical leads with one or more structures to reduce the likelihood of patients feeling electrical therapy delivered via the implantable medical leads.

### BACKGROUND

Sudden cardiac death (SCD) affects more than 300,000 patients annually and accounts for nearly 6% of the annual mortality in the U.S., the vast majority due to fatal arrhythmias. Implantable cardioverter defibrillators (ICDs) are used to prevent SCD by applying low voltage electrical stimulation (e.g., anti-tachycardia pacing [ATP]) and/or a high voltage (e.g., defibrillation) electrical shock to the heart when a life-threatening arrhythmia is detected. Endocardial delivery of ATP may minimize patient pain and discomfort by reducing the number of defibrillation shocks delivered. However, implantation of transvenous leads for intracardiac pacing and defibrillation is not always possible due to anatomic anomalies, occluded vessels, or other issues. For these patients, an extravascular ICD (EV-ICD) system may provide an opportunity to receive life-saving implantable pacing/defibrillation systems. An EV-ICD system may include an extravascular lead that may be implanted in the substernal space and a device implanted on the thorax to offer lower defibrillation thresholds (DFTs), reduced device complexity, size, and cost, and improved device longevity.

### SUMMARY

An EV-ICD system may convert lethal arrhythmias via ATP. However, the delivery of electrical therapy, such as pacing pulses, via electrodes of extravascular leads has resulted in patient feeling the electrical therapy, which reduces utilization of pacing therapies available from an EV ICD.

This disclosure describes implantable medical leads and implantable systems, such as ICD systems, utilizing the leads. For example, implantable medical leads, as described in this disclosure, may be configured to reduce sensations a patient may feel from electrical therapy being delivered via electrodes in the implantable medical lead. Although described herein primarily in the context of ICD systems, various aspects of the techniques of this disclosure may be applied to implantable systems other than ICD systems, including, but not limited to, bradycardia or cardiac resynchronization therapy (CRT) pacemaker systems, or partially implantable systems, such as temporary external pacemakers. Accordingly, implantable medical leads may be used in contexts other than that of ICD systems, both cardiac and non-cardiac. For example, implantable medical leads as described herein may be used with an extracardiac pacemaker system without defibrillation capabilities.

In one example, an implantable medical electrical lead comprises a lead body defining a proximal portion and a distal portion, wherein at least a part of the distal portion of the lead body defines a three-dimensional undulating configuration; a proximal end of the distal portion disposed adjacent to the three-dimensional undulating configuration; a first defibrillation electrode and a second defibrillation electrode disposed along the three-dimensional undulating configuration spaced apart from one another by a distance; and a pacing electrode disposed between the first defibrillation electrode and the second defibrillation electrode, the pacing electrode configured to perform at least one of delivering a pacing pulse to a heart and sensing cardiac electrical activity of the heart, wherein the first defibrillation electrode is disposed between the proximal end and the pacing electrode, a first portion of the first defibrillation electrode, a first portion of the second defibrillation electrode, and the pacing electrode are disposed along a plane, and a second portion of the first defibrillation electrode adjacent to the pacing electrode arcs in a direction orthogonal to the plane.

In another example, an implantable medical system includes an implantable medical device comprising a housing; and therapy delivery circuitry within the housing; and an implantable medical lead configured to be coupled to the medical device. The implantable medical lead comprising a lead body defining a proximal portion and a distal portion, wherein at least a part of the distal portion of the lead body defines a three-dimensional undulating configuration; a proximal end of the distal portion disposed adjacent to the three-dimensional undulating configuration; a first defibrillation electrode and a second defibrillation electrode disposed along the three-dimensional undulating configuration spaced apart from one another by a distance and disposed distally along the three-dimensional undulating configuration; and a pacing electrode disposed between the first defibrillation electrode and the second defibrillation electrode, the pacing electrode configured to perform at least one of delivering a pacing pulse to a heart and sensing cardiac electrical activity of the heart, wherein the first defibrillation electrode is disposed between the proximal end and the pacing electrode, a first portion of the first defibrillation electrode, a first portion of the second defibrillation electrode, and the pacing electrode are disposed along a plane, and a second portion of the first defibrillation electrode adjacent to the pacing electrode arcs in a direction orthogonal to the plane.

This summary is intended to provide an overview of the subject matter described in this disclosure. It is not intended to provide an exclusive or exhaustive explanation of the systems, devices, and methods described in detail within the accompanying drawings and description below. Further details of one or more examples are set forth in the accompanying drawings and the description below. Other features, objects, and advantages will be apparent from the description and drawings, and from the statements provided below.

### BRIEF DESCRIPTION OF DRAWINGS

FIG. 1A is a front view of a patient with an extracardiovascular ICD system implanted intra-thoracically.

FIG. 1B is a side view of the patient with the extracardiovascular ICD system implanted intra-thoracically.

FIG. 1C is a transverse view of the patient with the extracardiovascular ICD system implanted intra-thoracically.

FIGS. 2A-2C are conceptual diagrams of three different rotational views of an example distal portion of an example lead.

FIG. 2D is an image of an example distal portion of an example lead.

FIG. 3A is a conceptual diagram of an example extracardiovascular ICD system implanted.

FIG. 3B is an image of an example distal portion of a lead implanted.

FIG. 4 is a functional block diagram of an example configuration of electronic components of an example ICD.

### DETAILED DESCRIPTION

The delivery of electrical therapy, such as pacing pulses, via electrodes of extravascular leads has resulted in patient feeling the electrical therapy, which reduces utilization of pacing therapies available from an EV ICD.

In accordance with the techniques of the disclosure, an implantable medical electrical lead may be configured to decrease sensations a patient may feel from electrical therapy delivered via the implantable medical electrical lead. For example, an implantable medical electrical lead may include a lead body defining a proximal portion and a distal portion, wherein at least a part of the distal portion of the lead body defines a three-dimensional undulating configuration. The implantable medical electrical lead may further include a first defibrillation electrode and a second defibrillation electrode disposed along the three-dimensional undulating configuration spaced apart from one another by a distance, and a pacing electrode disposed between the first defibrillation electrode and the second defibrillation electrode, the pacing electrode configured to perform at least one of delivering a pacing pulse to a heart and sensing cardiac electrical activity of the heart. In some examples, an increased distance between defibrillation electrodes and a pacing electrode due to the three-dimensional undulating configuration of the distal portion may reduce a level of sensation felt by a patient when electrical therapy, such as pacing pulses, is applied to the patient.

In addition, a distal portion of the lead body having a three-dimensional undulating configuration in accordance with the techniques of the disclosure may also help the pacing electrode(s) and defibrillation electrode(s) of the implantable medical lead be positioned in a patient to reduce stimulation of extracardiac tissue, such as sensory or motor nerves, when electrical therapy, such as pacing pulses, is delivered via the pacing electrode. This may reduce pain or other sensations associated with stimulation of such tissue.

As used herein, relational terms, such as “first” and “second,” “over” and “under,” “front” and “rear,” and the like, may be used solely to distinguish one entity or element from another entity or element without necessarily requiring or implying any physical or logical relationship or order between such entities or elements.

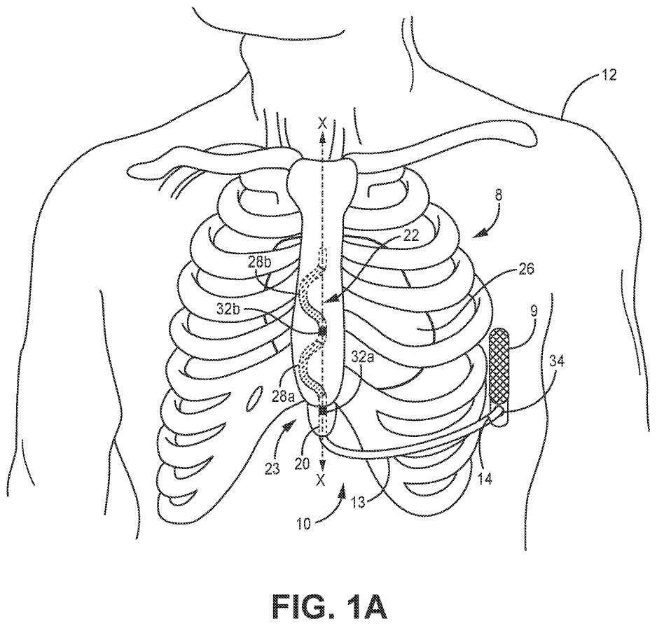

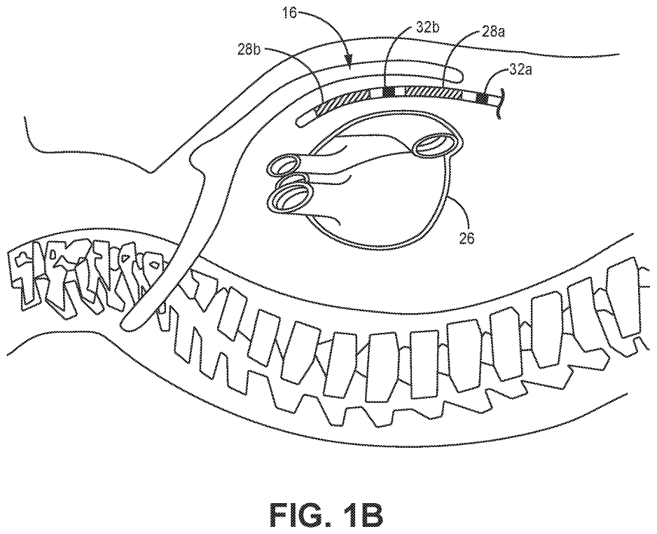

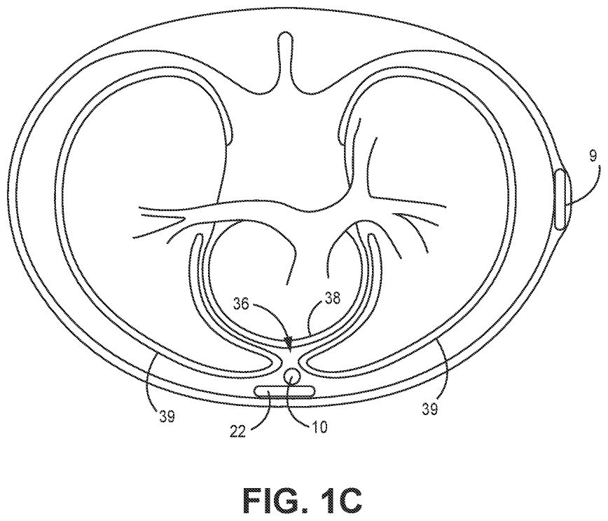

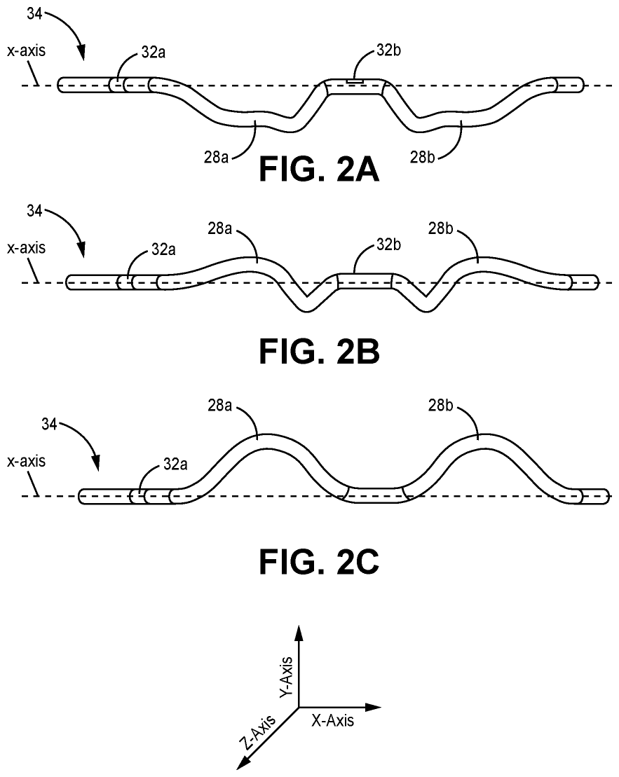

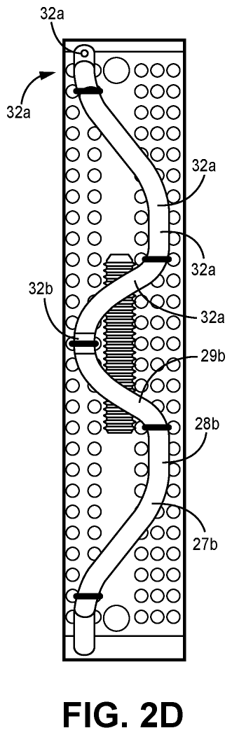

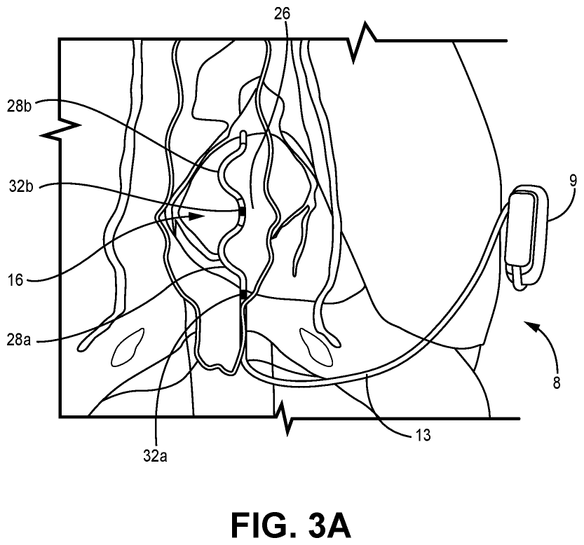

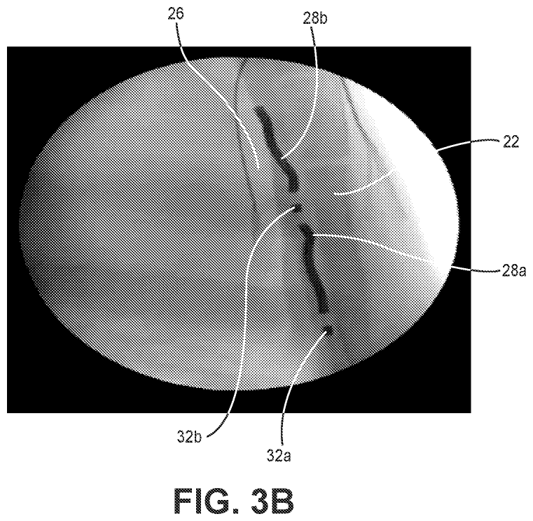

FIG. 1A is a front view of a patient 12 implanted with extracardiovascular ICD system 8 implanted intra-thoracically. Referring now to the drawings in which like reference designators refer to like elements, there is shown in FIGS. 1A-IC conceptual diagrams illustrating various views of an example extracardiovascular ICD system 8. ICD system 8 includes an ICD 9 connected to an implantable medical lead 10. FIG. 1A is a front view of a patient implanted with extracardiovascular ICD system 8. FIG. 1B is a side view of the patient implanted with extracardiovascular ICD system 8. FIG. 1C is a transverse view of the patient implanted with extracardiovascular ICD system 8. In some examples, distal portion 16 of implantable medical lead 10 may have a three-dimensional undulating shape and configuration, such as, but not limited to, the shapes and configurations shown in FIGS. 2A-2D and FIGS. 3A-3B.

ICD 9 may include a housing that forms a hermetic seal that protects components of the ICD 9. The housing of ICD 9 may be formed of a conductive material, such as titanium or titanium alloy, which may function as a housing electrode (sometimes referred to as a can electrode). In some embodiments, ICD 9 may be formed to have or may include a plurality of electrodes on the housing. ICD 9 may also include a connector assembly (also referred to as a connector block or header) that includes electrical feedthroughs through which electrical connections are made between conductors of lead 10 and electronic components included within the housing of ICD 9. As will be described in further detail herein, the housing may house one or more processors, memories, transmitters, receivers, sensors, sensing circuitry, therapy circuitry, power sources and other appropriate components. The housing is configured to be implanted in a patient, such as patient 12.

ICD 9 may be implanted extra-thoracically on the left side of the patient, e.g., under the skin and outside the ribcage (subcutaneously or submuscularly). ICD 9 may, in some instances, be implanted between the left posterior axillary line and the left anterior axillary line of the patient. ICD 9 may, however, be implanted at other extra-thoracic locations on the patient as described later.

Lead 10 may include an elongated lead body 13 having a distal portion 16 sized to be implanted in an extracardiovascular location proximate the heart, e.g., intra-thoracically, as illustrated in FIGS. 1A-1C, or extra-thoracically. For example, lead 10 may extend extra-thoracically under the skin and outside the ribcage (e.g., subcutaneously or submuscularly) from ICD 9 toward the center of the torso of the patient, for example, toward the xiphoid process 23 of the patient. At a position proximate xiphoid process 23, the lead body 13 may bend or otherwise turn and extend superiorly. The bend may be pre-formed and/or lead body 13 may be flexible to facilitate bending. In the example illustrated in FIGS. 1A-IC, the lead body 13 extends superiorly intra-thoracically underneath the sternum, in a direction substantially parallel to the sternum.

Distal portion 16 of lead 10 may reside in a substernal location such that distal portion 16 of lead 10 extends superior along the posterior side of the sternum substantially within the anterior mediastinum 36. Anterior mediastinum 36 may be viewed as being bounded laterally by pleurae 39, posteriorly by pericardium 38, and anteriorly by the sternum 22. In some instances, the anterior wall of anterior mediastinum 36 may also be formed by the transversus thoracis and one or more costal cartilages. Anterior mediastinum 36 includes a quantity of loose connective tissue (such as areolar tissue), adipose tissue, some lymph vessels, lymph glands, substernal musculature (e.g., transverse thoracic muscle), the thymus gland, branches of the internal thoracic artery, and the ITV.

Lead body 13 may extend superiorly extra-thoracically (instead of intra-thoracically), e.g., either subcutaneously or submuscularly above the ribcage/sternum. Lead 10 may be implanted at other locations, such as over the sternum, offset to the right of the sternum, angled lateral from the proximal or distal end of the sternum, or the like. In some examples, lead 10 may be implanted within an extracardiac vessel within the thorax, such as the ITV, the intercostal veins, the superior epigastric vein, or the azygos, hemiazygos, and accessory hemiazygos veins. In some examples, distal portion 16 of lead 10 may be oriented differently than is illustrated in FIGS. 1A-1C, such as orthogonal or otherwise transverse to sternum 22 and/or inferior to heart 26. In such examples, distal portion 16 of lead 10 may be at least partially within anterior mediastinum 36. In some examples, distal portion 16 of lead 10 may be placed between the heart and lung as well as within the pleural cavity.

Lead body 13 may have a generally tubular or cylindrical shape and may define a diameter of approximately 3-9 French (Fr). However, lead bodies of less than 3 Fr and more than 9 Fr may also be utilized. In another configuration, lead body 13 may have a flat, ribbon, or paddle shape with solid, woven filament, or metal mesh structure, along at least a portion of the length of the lead body 13. In such an example, the width across lead body 13 may be between 1-3.5 mm. Other lead body designs may be used without departing from the scope of this application.

Lead body 13 may be formed from a non-conductive material, including silicone, polyurethane, fluoropolymers, mixtures thereof, and other appropriate materials, and shaped to form one or more lumens (not shown), however, the techniques are not limited to such constructions. Distal portion 16 may be fabricated to be biased in a desired configuration, or alternatively, may be manipulated by the user into the desired configuration. For example, the distal portion 16 may be composed of a malleable material such that the user can manipulate the distal portion into a desired configuration where it remains until manipulated to a different configuration.

Lead body 13 may include a proximal portion 14 and a distal portion 16 which include electrodes configured to deliver electrical energy to the heart or sense electrical signals of the heart. Distal portion 16 may be anchored to a desired position within the patient, for example, substernally or subcutaneously by, for example, suturing distal portion 16 to the patient's musculature, tissue, or bone at the xiphoid process entry site. In some examples, distal portion 16 may be anchored to the patient or through the use of rigid tines, prongs, barbs, clips, screws, and/or other projecting elements or flanges, disks, pliant tines, flaps, porous structures such as a mesh-like elements and metallic or non-metallic scaffolds that facilitate tissue growth for engagement, bio-adhesive surfaces, and/or any other non-piercing elements.

Lead body 13 may define a substantially linear portion 20 (FIG. 1A) as it curves or bends near the xiphoid process 23 and extends superiorly. At least a part of distal portion 16 may define a three-dimensional undulating configuration distal to the substantially linear portion 20. In some examples, distal portion 16 may define a three-dimensional undulating pattern, e.g., zig-zag, meandering, sinusoidal, serpentine, or other pattern, as it extends toward the distal end of lead 10. In particular, as shown in FIGS. 2A-2D, distal portion 16 may include a three-dimensional undulating, meandering pattern. FIG. 2D shows an example configuration of distal portion 16. For example, a proximal end 34 of the distal portion disposed may be positioned adjacent to the three-dimensional undulating configuration. The first defibrillation electrode 28a may be disposed between the proximal end 34 and the pacing electrode 32b. In some examples, first portion 27a of the first defibrillation electrode 28a, first portion 27b of the second defibrillation electrode 28b, and pacing electrode 32b may be disposed along a plane. In some examples, proximal end 34 of the distal portion 16 may also be disposed along the plane. FIG. 2D shows an example of the distal portion of an example lead being arranged on a board. In some examples, as shown in FIG. 2D, a second portion 29a of the first defibrillation electrode 28a adjacent to the pacing electrode 32b may arc in a direction orthogonal to the plane. In some examples, as shown in FIG. 2D, a second portion 29b of the second defibrillation electrode 28b adjacent to the pacing electrode 32b may arc in a direction orthogonal to the plane. In some examples, second portion 29a and second portion 29b may arc in the same orthogonal direction. In some examples, second portion 29a and second portion 29b may arc in an opposite orthogonal direction to each other. In other configurations, lead body 13 may not have a substantially linear portion 20 as it extends superiorly, but instead the three-dimensional undulating configuration may begin immediately after the bend.

Distal portion 16 includes one or more defibrillation electrodes configured to deliver an anti-tachyarrhythmia, e.g., cardioversion/defibrillation, shock to heart 26 of patient 12. In some examples, distal portion 16 includes a plurality of defibrillation electrodes spaced a distance apart from each other along the length of distal portion 16. In examples illustrated by FIGS. 1A-1C, FIGS. 2A-2D, and FIGS. 3A-3B, distal portion 16 includes two defibrillation electrodes 28a and 28b (collectively, “defibrillation electrodes 28”).

Defibrillation electrodes 28 may be disposed around or within the lead body 13 of the distal portion 16, or alternatively, may be embedded within the wall of the lead body 13. In one configuration, defibrillation electrodes 28 may be coil electrodes formed by a conductor. The conductor may be formed of one or more conductive polymers, ceramics, metal-polymer composites, semiconductors, metals or metal alloys, including but not limited to, one of a combination of the platinum, tantalum, titanium, niobium, zirconium, ruthenium, indium, gold, palladium, iron, zinc, silver, nickel, aluminum, molybdenum, stainless steel, MP35N, carbon, copper, polyaniline, polypyrrole, and other polymers. In another configuration, each of defibrillation electrodes 28 may be a flat ribbon electrode, a paddle electrode, a braided or woven electrode, a mesh electrode, a directional electrode, a patch electrode or another type of electrode configured to deliver a cardioversion/defibrillation shock to heart 26 of patient 12.

Defibrillation electrodes 28 may be electrically connected to one or more conductors, which may be disposed in the body wall of lead body 13 or in one or more insulated lumens (not shown) defined by lead body 13. In an example configuration, each of defibrillation electrodes 28 is connected to a common conductor such that a voltage may be applied simultaneously to all defibrillation electrodes 28 to deliver an anti-tachyarrhythmia shock to heart 26. In other configurations, defibrillation electrodes 28 may be attached to separate conductors such that each defibrillation electrode 28 may apply a voltage independent of the other defibrillation electrodes 28. In this case, ICD 9 or lead 10 may include one or more switches or other mechanisms to electrically connect the defibrillation electrodes together to function as a common polarity electrode such that a voltage may be applied simultaneously to all defibrillation electrodes 28 in addition to being able to independently apply a voltage.

Distal portion 16 may also include one or more pacing and/or sensing electrodes configured to deliver pacing pulses to heart 26 and/or sense electrical activity of heart 26. Such electrodes may be referred to as pacing electrodes, sensing electrodes, or pacing/sensing electrodes. In examples illustrated by FIGS. 1A-IC, FIGS. 2A-2D, and FIGS. 3A-3B, distal portion 16 includes two pacing/sensing electrodes 32a and 32b (collectively, “pacing/sensing electrodes 32”).

In the illustrated examples of FIGS. 1A-IC, FIGS. 2A-2D, and FIGS. 3A-3B, pacing/sensing electrode 32b is positioned between defibrillation electrodes 28, e.g., within a gap between the defibrillation electrodes, and pacing/sensing electrode 32a is positioned more proximal along distal portion 16 than proximal defibrillation electrode 28a. In some examples, more than one electrode may exist within the gap between defibrillation electrodes 28. In some examples, an electrode is additionally or alternatively located distal of the distalmost defibrillation electrode 28b.

In some examples, electrodes 32 may be configured to deliver low-voltage electrical pulses to the heart or may sense a cardiac electrical activity, e.g., depolarization and repolarization of the heart. As such, electrodes 32 may be referred to herein as pacing/sensing electrodes 32. In one configuration, electrodes 32 are ring electrodes. However, in other configurations electrodes 32 may be any of a number of different types of electrodes, including ring electrodes, short coil electrodes, paddle electrodes, hemispherical electrodes, electrode segments extending circumferentially around less than half of a circumference of the lead body, or directional electrodes. Each of electrodes 32 may be the same or different types of electrodes as others of electrodes 32. Electrodes 32 may be electrically isolated from an adjacent defibrillation electrode 28 by including an electrically insulating layer of material between electrodes 32 and adjacent defibrillation electrodes 28. Each electrode 32 may have its own separate conductor such that a voltage may be applied to or sensed via each electrode independently from another electrode 32.

Electrodes 28 are referred to as defibrillation electrodes, and electrodes 32 are referred to as pacing/sensing electrodes, because they may have different physical structures enabling different functionality. Defibrillation electrodes 28 may be larger, e.g., have greater surface area, than pacing/sensing electrodes 32 and, consequently, may be configured to deliver anti-tachyarrhythmia shocks that have relatively higher voltages than pacing pulses. The relatively smaller size of pacing/sensing electrodes 32 may provide advantages over defibrillation electrodes for delivering pacing pulses and sensing intrinsic cardiac activity, e.g., lower pacing capture thresholds and/or better sensed signal quality. Nevertheless, a defibrillation electrode 28 may be used to deliver pacing pulses and/or sense electrical activity of the heart, such as in combination with a pacing/sensing electrode 32.

In some examples, such as shown in FIGS. 1A-IC, FIGS. 2A-2D, and FIGS. 3A-3B, each electrode 32 may be substantially aligned along and/or near a major longitudinal axis (“x”). In one example, the major longitudinal axis is defined by a portion of elongate body 12, e.g., substantially linear portion 20. In another example, the major longitudinal axis is defined relative to the body of the patient, e.g., along the anterior median line (or midsternal line), one of the sternal lines (or lateral sternal lines), left parasternal line, or other line.

In one configuration, the midpoint of each electrode 32a and 32b is along the major longitudinal axis “x,” such that each electrode 32a and 32b is at least disposed at substantially the same horizontal position when the distal portion is implanted within the patient. In some examples, the longitudinal axis “x” may correspond to a caudal-cranial axis of the patient and a horizontal axis orthogonal to the longitudinal axis “x” may correspond to a medial-lateral axis of the patient. In other configurations, the electrodes 32 may be disposed at any longitudinal or horizontal position along the distal portion 16 disposed between, proximal to, or distal to the defibrillation electrodes 28. In the example illustrated in FIG. 1A, electrodes 32 are disposed along the undulating configuration of distal portion 16 at locations that will be closer to heart 26 of patient 12 than defibrillation electrodes 28 (e.g., at a peak of the undulating configuration that is toward the left side of the sternum). As illustrated in FIG. 1A, for example, electrodes 32 are substantially aligned with one another along the left sternal line. In the example illustrated in FIG. 1A, defibrillation electrodes 28 are disposed along peaks of the undulating configuration that extend toward a right side of the sternum away from the heart. This configuration places pacing/sensing electrodes 32 at locations closer to the heart than electrodes 28, to facilitate cardiac pacing and sensing at relatively lower amplitudes.

In some examples, the three-dimensional undulating configuration may not be substantially disposed in a common plane. For example, distal portion 16 may define a concavity or a curvature.

Proximal portion 14 of lead body 13 may include one or more connectors 34 to electrically couple lead 10 to ICD 9. ICD 9 may also include a connector assembly that includes electrical feedthroughs through which electrical connections are made between the one or more connectors 34 of lead 10 and the electronic components included within the housing. The housing of ICD 9 may house one or more processors, memories, transmitters, receivers, sensors, sensing circuitry, therapy circuitry, power sources (e.g., capacitors and batteries), and/or other components. The components of ICD 9 may generate and deliver electrical therapy such as anti-tachycardia pacing, cardioversion or defibrillation shocks, post-shock pacing, and/or bradycardia pacing.

The three-dimensional undulating configuration of distal portion 16 and the inclusion of electrodes 32 between defibrillation electrodes 28 may provide a number of therapy vectors for the delivery of electrical therapy to the heart. For example, at least a portion of defibrillation electrodes 28 and one of electrodes 32 may be disposed over the right ventricle, or any chamber of the heart, such that pacing pulses and anti-tachyarrhythmia shocks may be delivered to the heart. The housing of ICD 9 may be charged with or function as a polarity different than the polarity of the one or more defibrillation electrodes 28 and/or electrodes 32 such that electrical energy may be delivered between the housing and the defibrillation electrode 28 and/or electrode 32 to the heart.

Each defibrillation electrode 28 may have the same polarity as every other defibrillation electrode 28 when a voltage is applied to it such that a shock may be delivered from all defibrillation electrodes together. In examples in which defibrillation electrodes 28 are electrically connected to a common conductor within lead body 13, this is the configuration of defibrillation electrodes 28. However, in other examples, defibrillation electrodes 28 may be coupled to separate conductors within lead body 13 and may therefore each have different polarities such that electrical energy may flow between defibrillation electrodes 28, or between one of defibrillation electrodes 28 and one of pacing/sensing electrodes 32 or the housing electrode, to provide anti-tachyarrhythmia shock, pacing therapy, and/or to sense cardiac depolarizations. In this case, defibrillation electrodes 28 may still be electrically coupled together, e.g., via one or more switches within ICD 9, to have the same polarity.

The techniques of this disclosure may be applied to implantable systems other than ICD 9, including, but not limited to, bradycardia pacemaker systems, as well as with external stimulation devices, such as permanent or temporary external pacemakers or defibrillators.

In some examples, lead 10 may include a first defibrillation electrode 28a and a second defibrillation electrode 28b that are configured to deliver anti tachyarrhythmia shocks. In this example, pacing electrode 32b may be configured to deliver a pacing pulse that generates an electric field proximate to the pacing electrode.

FIGS. 2A-2C are conceptual diagrams of three different rotational views of an example distal portion 16 of lead 10. The distal portion 16, as shown in FIGS. 2A-2C, is shown at three different rotations about an x-axis. In some examples, the x-axis in FIGS. 2A-2C may correspond to the major longitudinal axis in FIGS. 1A-IC.

FIGS. 2A-2C are three views of an example 3D shape of distal portion 16 of lead body 13. For example, distal portion 16 may have a three-dimensional undulating shape, e.g., zig-zag, meandering, sinusoidal, serpentine, or other pattern, as it extends from proximal to distal ends (e.g., from pacing electrode 32a at a proximal end 34 to the opposite distal end). In some examples, pacing electrode 32a may be a ring electrode as shown, or may be a segmented electrode, e.g., segmented about the circumference of the lead body. In some examples, pacing electrode 32b may be a segment electrode, as shown, a ring electrode, a short coil electrode, a paddle electrode, a hemispherical electrode, or a directional electrode.

In some examples, when electrical therapy is applied to a pacing electrode, current may flow between neighboring defibrillation electrodes and the pacing electrode which may cause discomfort for a patient. For example, current flowing between pacing electrode 32b and one or both of defibrillation electrodes 28a and 28b may cause discomfort for a patient. The discomfort may be cause by a relatively denser current field at the edge(s) of the defibrillation electrode(s) proximate to the pacing electrode.

In some examples, as shown in FIGS. 2A-2D, a distal portion 16 may have a three-dimensional undulating shape in which second portions 29a, 29b of the respective defibrillation electrodes 28a, 28b undulate away from pacing electrode 32b in an orthogonal direction from plane including proximal end 34 and the pacing electrode 32b to increase separation distance of the pacing electrode 32b and second portions 29a, 29b of the respective defibrillation electrode 28a, 28b from the sternum. In some examples, distal portion 16 having a three-dimensional undulating shape described above may move the pacing electrode 32b and the proximate edges of defibrillation electrodes 28a, 28b more towards the heart. In some examples, distal portion 16 having a three-dimensional undulating shape described above may reduce a level of pacing sensation felt by a patient.

In some examples, proximal end 34 and pacing electrodes 32a, 32b may be substantially aligned on or near the axis, such as x-axis, and may also be substantially aligned on or near a plane, such as an x-y plane. In some examples, a surface of pacing electrode 32b may be arced and/or curved. In some examples, a surface of one or more of defibrillation electrodes 28a, 28b may be arced and/or curved. In some examples, as shown in FIGS. 2A and 2C, a longitudinal axis, such as the x-axis, may pass through pacing electrode 32a and pacing electrode 32, and defibrillation electrodes 28a, 28b may be positioned apart from the longitudinal axis.

FIG. 2D is an image of an example distal portion 16 of an example lead. As shown in FIG. 2D, a proximal end 34 of the distal portion disposed may be positioned adjacent to the three-dimensional undulating configuration. The first defibrillation electrode 28a may be disposed between the proximal end 34 and the pacing electrode 32b. In some examples, first portion 27a of the first defibrillation electrode 28a, first portion 27b of the second defibrillation electrode 28b, and pacing electrode 32b may be disposed along a plane. In some examples, proximal end 34 of the distal portion may also be disposed along the plane. FIG. 2D shows an example of the distal portion of an example lead being arranged on a board. In some examples, as shown in FIG. 2D, a second portion 29a of the first defibrillation electrode 28a adjacent to the pacing electrode 32b may are in a direction orthogonal to the plane. In some examples, as shown in FIG. 2D, a second portion 29b of the second defibrillation electrode 28b adjacent to the pacing electrode 32b may arc in a direction orthogonal to the plane. In some examples, second portion 29a and second portion 29b may arc in the same orthogonal direction. In some examples, second portion 29a and second portion 29b may arc in an opposite orthogonal direction. In some examples, proximal end may include pacing electrode 32a.

In some examples, second portion 29a of the first defibrillation electrode 28a arcing in a direction orthogonal to the plane may provide greater anchoring stability for the distal portion 16. In addition, second portion 29a of the first defibrillation electrode 28a arcing in a direction orthogonal to the plane may increase a distance between one or more of pacing electrode 32b and second portion 29a to the sternum, which may reduce a level of sensation, such as discomfort, felt by a patient when electrical therapy is applied to the pacing electrode 32b. Similarly, second portion 29b of the second defibrillation electrode 28b adjacent to the pacing electrode 32b arcing in a direction orthogonal to the plane may increase distance between one or more of pacing electrode 32b and second portion 29b to the sternum, which may reduce a level of sensation, such as discomfort, felt by a patient when electrical therapy is applied to the pacing electrode 32b.

Pacing electrode 32b may be positioned between the defibrillation electrodes 28a, 28b, and may or may not be “in line” with pacing electrode 32a and/or the distal end (which is shown without an electrode on the right side of lead body 13). The three-dimensional undulating shape may extend in or out of the page as shown, e.g., in the “z” direction. For example, the three-dimensional undulating shape may be a 3D curve, (e.g., helix, spiral, or other 3D curve and/or parametric curve).

In some examples, three-dimensional shape of distal portion 16 of lead body 13 and/or pacing electrode 32b being segmented may increase the separation between the interior surface of thoracic wall end areas which may have high electrical current gradients near pacing electrode 32b (e.g. cathode) and adjacent edges of the defibrillation electrodes 28a, 28b (e.g. anode). In some examples, increased distance between defibrillation electrodes 28a, 28b and pacing electrode 32b, as shown in FIGS. 2A-2D, may reduce a level of sensation felt by a patient when electrical therapy is applied to the patient via electrode(s), such as pacing electrode 32b.

In some examples, pacing electrode 32b being segmented may increase the electrical separation between the interior surface of thoracic wall end areas which may have high electrical current gradients near pacing electrode 32b (e.g. cathode) and adjacent edges, such as second portions 29a, 29b, of the defibrillation electrodes 28a, 28b (e.g. anode). In some examples, increased electrical separation between second portions 29a, 29b of respective defibrillation electrodes 28a, 28b and pacing electrode 32b may also reduce a level of sensation felt by a patient when electrical therapy is applied to the patient via electrode(s), such as pacing electrode 32b.

In some examples, distal portion 16 may be fabricated to be biased in a desired configuration, or alternatively, may be manipulated by the user into the desired configuration, as shown in FIGS. 2A-2D. For example, the distal portion 16 may be composed of a malleable material such that the user can manipulate the distal portion into a desired configuration where it remains until manipulated to a different configuration. In some examples, distal portion 16 may be manipulated to be straight as lead 10 is introduced via implant tool (e.g., via a sheath). Distal portion 16 may take on its pre-formed three-dimensional undulating configuration after the distal portion 16 is implanted and the implant tool is withdrawn.

In some examples, lead 10 may include one or more markers (not shown) to indicate an orientation of lead 10. In some examples, distal portion 16 may include one or more markers to indicate an orientation of distal portion 16. In some examples, the one or more markers may indicate an orientation of one or more of defibrillation electrode 28a, defibrillation electrode 28b, pacing electrode 32b, or pacing electrode 32a. In some examples, the one or more markers may be a radiopaque marker. In some examples, the one or more markers may be other types of markers as well or any combination of radiopaque marker and other types of markers.

FIG. 3A is a conceptual diagram of an example ICD system 8 being implanted. In some examples, ICD system 8 may have a distal portion 16 being shaped as shown in FIGS. 2A-2C. In the example, the three-dimensional undulating shape of the distal portion 16 may help pacing electrode 32b to be positioned adjacent to or against heart 26 and away from the sternum, while defibrillation electrodes 28a, 28b are to be positioned adjacent to or against the sternum. The positioning of defibrillation electrodes 28a, 28b and pacing electrodes 32a, 32b in the distal portion 16, as shown in FIG. 3A, may reduce a level of sensation felt by a patient when electrical therapy is applied to the patient.

FIG. 3B is an image of an example of distal portion 16 implanted. In some examples, distal portion 16 may be shaped as shown in FIGS. 2A-2C. As shown in FIG. 3B, the three-dimensional undulating shape of the distal portion 16 helps defibrillation electrodes 28a, 28b to be positioned against or adjacent to sternum 22, and away from heart 26, and helps pacing electrode 32b to be positioned against or adjacent to heart 26, and away from the sternum 22. The positioning of defibrillation electrodes 28a, 28b and pacing electrodes 32a, 32b in distal portion 16, as shown in FIG. 3B, may reduce a level of sensation felt by a patient when electrical therapy is applied to the patient.

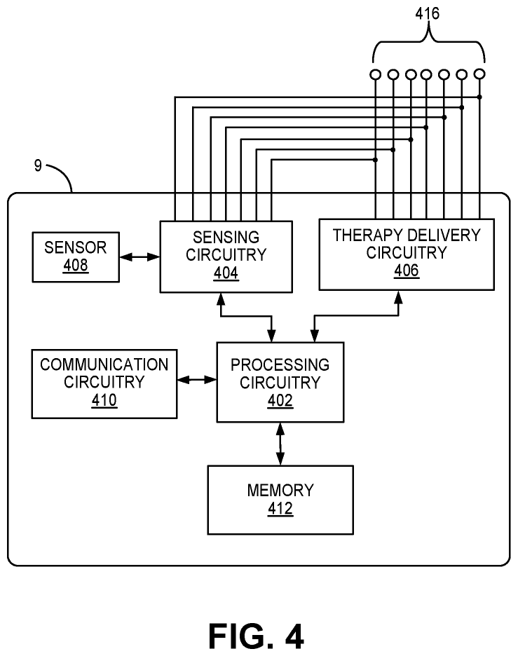

FIG. 4 is a functional block diagram of an example configuration of electronic components and other components of ICD 9. ICD 9 includes a processing circuitry 402, sensing circuitry 404, therapy delivery circuitry 406, sensors 408, communication circuitry 410, and memory 412. In some examples, ICD 9 may include more or fewer components. The described circuitry and other components may be implemented together on a common hardware component or separately as discrete but interoperable hardware or software components. Depiction of different features is intended to highlight different functional aspects and does not necessarily imply that such circuitry and other components must be realized by separate hardware or software components. Rather, functionality associated with one or more circuitries and components may be performed by separate hardware or software components, or integrated within common or separate hardware or software components.

Sensing circuitry 404 may be electrically coupled to some or all of electrodes 416, which may correspond to any of the defibrillation, pacing/sensing, and housing electrodes described herein. Sensing circuitry 404 is configured to obtain signals sensed via one or more combinations of electrodes 416 and process the obtained signals.

The components of sensing circuitry 404 may be analog components, digital components or a combination thereof. Sensing circuitry 404 may, for example, include one or more sense amplifiers, filters, rectifiers, threshold detectors, analog-to-digital converters (ADCs) or the like. Sensing circuitry 404 may convert the sensed signals to digital form and provide the digital signals to processing circuitry 402 for processing or analysis. For example, sensing circuitry 404 may amplify signals from the sensing electrodes and convert the amplified signals to multi-bit digital signals by an ADC. Sensing circuitry 404 may also compare processed signals to a threshold to detect the existence of atrial or ventricular depolarizations (e.g., P- or R waves) and indicate the existence of the atrial depolarization (e.g., P-waves) or ventricular depolarizations (e.g., R-waves) to processing circuitry 402. As shown in FIG. 4, ICD 9 may additionally include one or more sensors 408, such as one or more accelerometers, which may be configured to provide signals indicative of other parameters of a patient, such as activity or posture, to processing circuitry 402.

Processing circuitry 402 may process the signals from sensing circuitry 404 to monitor electrical activity of heart 26 of patient 12. Processing circuitry 402 may store signals obtained by sensing circuitry 404 as well as any generated EGM waveforms, marker channel data or other data derived based on the sensed signals in memory 412. Processing circuitry 402 may analyze the EGM waveforms and/or marker channel data to detect arrhythmias (e.g., bradycardia or tachycardia). In response to detecting the cardiac event, processing circuitry 402 may control therapy delivery circuitry 406 to deliver the desired therapy to treat the cardiac event, e.g., defibrillation shock, cardioversion shock, ATP, post shock pacing, or bradycardia pacing.

Therapy delivery circuitry 406 is configured to generate and deliver electrical therapy to heart 26. Therapy delivery circuitry 406 may include one or more pulse generators, capacitors, and/or other components capable of generating and/or storing energy to deliver as pacing therapy, defibrillation therapy, cardioversion therapy, cardiac resynchronization therapy, other therapy or a combination of therapies. In some instances, therapy delivery circuitry 406 may include a first set of components configured to provide pacing therapy and a second set of components configured to provide defibrillation therapy. In some instances, therapy delivery circuitry 406 may utilize the same set of components to provide both pacing and defibrillation therapy. In still other instances, therapy delivery circuitry 406 may share some of the defibrillation and pacing therapy components while using other components solely for defibrillation or pacing. Processing circuitry 402 may control therapy delivery circuitry 406 to deliver the generated therapy to heart 26 via one or more combinations of electrodes 416. Although not shown in FIG. 4, ICD 9 may include switching circuitry configurable by processing circuitry 402 to control which of electrodes 416 is connected to therapy delivery circuitry 406 and sensing circuitry 404.

Communication circuitry 410 may include any suitable hardware, firmware, software or any combination thereof for communicating with another device, such as a clinician programmer, a patient monitoring device, or the like. For example, communication circuitry 410 may include appropriate modulation, demodulation, frequency conversion, filtering, and amplifier components for transmission and reception of data with the aid of an antenna.

The various components of ICD 9 may include any one or more processors, controllers, digital signal processors (DSPs), application specific integrated circuits (ASICs), field-programmable gate arrays (FPGAs), or equivalent discrete or integrated circuitry, including analog circuitry, digital circuitry, or logic circuitry. Processing circuitry 402 may include fixed function circuitry and/or programmable processing circuitry. The functions attributed to processing circuitry 402 herein may be embodied as software, firmware, hardware or any combination thereof.

Memory 412 may include computer-readable instructions that, when executed by processing circuitry 402 or other components of ICD 9, cause one or more components of ICD 9 to perform various functions attributed to those components in this disclosure. Memory 412 may include any volatile, non-volatile, magnetic, optical, or electrical media, such as a random-access memory (RAM), read-only memory (ROM), non-volatile RAM (NVRAM), static non-volatile RAM (SRAM), electrically-erasable programmable ROM (EEPROM), flash memory, or any other non-transitory computer-readable storage media.

The leads and systems described herein may be used at least partially within the substernal space, e.g., within anterior mediastinum of patient, to provide an extravascular ICD system. An implanter (e.g., a physician) may implant the distal portion of the lead intra-thoracically using any of a number of implant tools, e.g., tunneling rod, sheath, or other tool that can traverse the diagrammatic attachments and form a tunnel in the substernal location. For example, the implanter may create an incision near the center of the torso of the patient, e.g., and introduce the implant tool into the substernal location via the incision. The implant tool is advanced from the incision superior along the posterior of the sternum in the substernal location. The distal portion of the lead is introduced into the tunnel via implant tool (e.g., via a sheath). As the distal portion is advanced through the substernal tunnel, the distal portion is relatively straight. The pre-formed or shaped three-dimensional undulating configuration is flexible enough to be straightened out while routing the lead through a sheath or other lumen or channel of the implant tool. Once the distal portion is in place, the implant tool is withdrawn toward the incision and removed from the body of the patient while leaving the lead in place along the substernal path. As the implant tool is withdrawn, the distal end of the lead takes on its pre-formed three-dimensional undulating configuration. In some examples, rather than extending in a superior direction along the sternum, the distal portion of the lead may be oriented orthogonal or otherwise transverse to the sternum and/or inferior to the heart.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module, unit, or circuit for purposes of clarity, it should be understood that the techniques of this disclosure may be performed by a combination of units, modules, or circuitry associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processing circuitry” as used herein may refer to any of the foregoing structure or any other physical structure suitable for implementation of the described techniques. Also, the techniques could be fully implemented in one or more circuits or logic elements.

The following examples are illustrative of the techniques described herein.

Example 1: An implantable medical electrical lead includes a lead body defining a proximal portion and a distal portion, wherein at least a part of the distal portion of the lead body defines a three-dimensional undulating configuration; a proximal end of the distal portion disposed adjacent to the three-dimensional undulating configuration; a first defibrillation electrode and a second defibrillation electrode disposed along the three-dimensional undulating configuration spaced apart from one another by a distance; and a pacing electrode disposed between the first defibrillation electrode and the second defibrillation electrode, the pacing electrode configured to perform at least one of delivering a pacing pulse to a heart and sensing cardiac electrical activity of the heart, wherein the first defibrillation electrode is disposed between the proximal end and the pacing electrode, a first portion of the first defibrillation electrode, a first portion of the second defibrillation electrode, and the pacing electrode are disposed along a plane, and a second portion of the first defibrillation electrode adjacent to the pacing electrode arcs in a direction orthogonal to the plane.

Example 2: The implantable medical lead of example 1, wherein the first portion of the first defibrillation electrode, the first portion of the second defibrillation electrode, the proximal end of the distal portion, and the pacing electrode are disposed along the plane.

Example 3: The implantable medical lead of any of examples 1 and 2, wherein a second portion of the second defibrillation electrode adjacent to the pacing electrode arcs in a direction orthogonal to the plane.

Example 4: The implantable medical lead of any of examples 1-3, wherein the three-dimensional undulating configuration defines a three-dimensional parametric curve.

Example 5: The implantable medical lead of any of examples 1-4, wherein the pacing electrode is segmented.

Example 6: The implantable medical lead of example 5, wherein the pacing electrode is positioned about the circumference of the lead body.

Example 7: The implantable medical lead of any of examples 1 through 6, wherein a mask is disposed at least over a portion of an outer surface of the pacing electrode.

Example 8: The implantable medical lead of any of examples 1-7, wherein the proximal end comprises a second pacing electrode, wherein the second pacing electrode is configured to perform at least one of delivering a pacing pulse to a heart and sensing cardiac electrical activity of the heart, and the first defibrillation electrode is disposed between the pacing electrode and the second pacing electrode.

Example 9: The implantable medical lead of example 8, wherein a longitudinal axis passes through the pacing electrode and the second pacing electrode, and the first defibrillation electrode and the second defibrillation electrode disposed along the three-dimensional undulating configuration are positioned apart from the longitudinal axis.

Example 10: The implantable medical lead of any of examples 1-9, wherein the first defibrillation electrode and the second defibrillation electrode are configured to be positioned adjacent to a sternum or to contact the sternum.

Example 11: The implantable medical lead of any of examples 9 and 10, wherein the first defibrillation electrode and the second defibrillation electrode are configured to contact the sternum, wherein the contact with the sternum is to cause the pacing electrode to be positioned away from the sternum and towards the heart.

Example 12: The implantable medical lead of any of examples 1-11, wherein the pacing electrode is configured to contact the heart or to be positioned adjacent to the heart.

Example 13: The implantable medical lead of example 12, wherein the second pacing electrode is configured to contact the heart or positioned adjacent to the heart.

Example 14: The implantable medical lead of any of examples 1-13, further comprising a marker to indicate orientation of the lead.

Example 15: The implantable medical lead of example 14, wherein the marker indicates orientation of the pacing electrode.

Example 16: The implantable medical lead of any of examples 14-15, wherein the marker is a radiopaque marker.

Example 17: An implantable medical system includes an implantable medical device includes a housing; and therapy delivery circuitry within the housing; and an implantable medical lead configured to be coupled to the medical device includes a lead body defining a proximal portion and a distal portion, wherein at least a part of the distal portion of the lead body defines a three-dimensional undulating configuration; a proximal end of the distal portion disposed adjacent to the three-dimensional undulating configuration; a first defibrillation electrode and a second defibrillation electrode disposed along the three-dimensional undulating configuration spaced apart from one another by a distance and disposed distally along the three-dimensional undulating configuration; and a pacing electrode disposed between the first defibrillation electrode and the second defibrillation electrode, the pacing electrode configured to perform at least one of delivering a pacing pulse to a heart and sensing cardiac electrical activity of the heart, wherein the first defibrillation electrode is disposed between the proximal end and the pacing electrode, a first portion of the first defibrillation electrode, a first portion of the second defibrillation electrode, and the pacing electrode are disposed along a plane, and a second portion of the first defibrillation electrode adjacent to the pacing electrode arcs in a direction orthogonal to the plane.

Example 18: The implantable medical system of example 16, comprising the implantable medical lead of any of examples 2-16.

It will be appreciated by persons skilled in the art that the present application is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the application, which is limited only by the following claims.

1. An implantable medical electrical lead comprising:
a lead body defining a proximal portion and a distal portion, wherein at least a part of the distal portion of the lead body defines a three-dimensional undulating configuration;
a proximal end of the distal portion disposed adjacent to the three-dimensional undulating configuration;
a first defibrillation electrode and a second defibrillation electrode disposed along the three-dimensional undulating configuration spaced apart from one another by a distance; and
a pacing electrode disposed between the first defibrillation electrode and the second defibrillation electrode, the pacing electrode configured to perform at least one of delivering a pacing pulse to a heart and sensing cardiac electrical activity of the heart,
wherein the first defibrillation electrode is disposed between the proximal end and the pacing electrode,
a first portion of the first defibrillation electrode, a first portion of the second defibrillation electrode, and the pacing electrode are disposed along a plane, and
a second portion of the first defibrillation electrode adjacent to the pacing electrode arcs in a direction orthogonal to the plane.

2. The implantable medical lead of claim 1, wherein the first portion of the first defibrillation electrode, the first portion of the second defibrillation electrode, the proximal end of the distal portion, and the pacing electrode are disposed along the plane.

3. The implantable medical lead of claim 1, wherein a second portion of the second defibrillation electrode adjacent to the pacing electrode arcs in a direction orthogonal to the plane.

4. The implantable medical lead of claim 1, wherein the three-dimensional undulating configuration defines a three-dimensional parametric curve.

5. The implantable medical lead of claim 1, wherein the pacing electrode is segmented.

6. The implantable medical lead of claim 5, wherein the pacing electrode is positioned about the circumference of the lead body.

7. The implantable medical lead of claim 1, wherein a mask is disposed at least over a portion of an outer surface of the pacing electrode.

8. The implantable medical lead of claim 1, wherein the proximal end comprises a second pacing electrode, wherein the second pacing electrode is configured to perform at least one of delivering a pacing pulse to a heart and sensing cardiac electrical activity of the heart, and the first defibrillation electrode is disposed between the pacing electrode and the second pacing electrode.

9. The implantable medical lead of claim 8, wherein a longitudinal axis passes through the pacing electrode and the second pacing electrode, and the first defibrillation electrode and the second defibrillation electrode disposed along the three-dimensional undulating configuration are positioned apart from the longitudinal axis.

10. The implantable medical lead of claim 1, wherein the first defibrillation electrode and the second defibrillation electrode are configured to be positioned adjacent to a sternum or to contact the sternum.

11. The implantable medical lead of claim 10, wherein the first defibrillation electrode and the second defibrillation electrode are configured to contact the sternum, wherein the contact with the sternum is to cause the pacing electrode to be positioned away from the sternum and towards the heart.

12. The implantable medical lead of claim 1, wherein the pacing electrode is configured to contact the heart or to be positioned adjacent to the heart.

13. The implantable medical lead of claim 12, wherein the second pacing electrode is configured to contact the heart or positioned adjacent to the heart.

14. The implantable medical lead of claim 1, further comprising a marker to indicate orientation of the lead.

15. The implantable medical lead of claim 14, wherein the marker indicates orientation of the pacing electrode.

16. The implantable medical lead of claim 1, wherein the marker is a radiopaque marker.

17. An implantable medical system comprising:
an implantable medical device comprising:
a housing; and
therapy delivery circuitry within the housing; and
an implantable medical lead configured to be coupled to the medical device comprising:
a lead body defining a proximal portion and a distal portion, wherein at least a part of the distal portion of the lead body defines a three-dimensional undulating configuration;
a proximal end of the distal portion disposed adjacent to the three-dimensional undulating configuration;
a first defibrillation electrode and a second defibrillation electrode disposed along the three-dimensional undulating configuration spaced apart from one another by a distance and disposed distally along the three-dimensional undulating configuration; and
a pacing electrode disposed between the first defibrillation electrode and the second defibrillation electrode, the pacing electrode configured to perform at least one of delivering a pacing pulse to a heart and sensing cardiac electrical activity of the heart,
wherein the first defibrillation electrode is disposed between the proximal end and the pacing electrode,
a first portion of the first defibrillation electrode, a first portion of the second defibrillation electrode, and the pacing electrode are disposed along a plane, and
a second portion of the first defibrillation electrode adjacent to the pacing electrode arcs in a direction orthogonal to the plane.

18. The implantable medical system of claim 17, wherein the first portion of the first defibrillation electrode, the first portion of the second defibrillation electrode, the proximal end of the distal portion, and the pacing electrode are disposed along the plane.

19. The implantable medical system of claim 17, wherein a second portion of the second defibrillation electrode adjacent to the pacing electrode arcs in a direction orthogonal to the plane.

20. The implantable medical system of claim 17, wherein the proximal end comprises a second pacing electrode, wherein the second pacing electrode is configured to perform at least one of delivering a pacing pulse to a heart and sensing cardiac electrical activity of the heart, and the first defibrillation electrode is disposed between the pacing electrode and the second pacing electrode.
